(19) United States
(12) Patent Application Publication
(10) Pub. No.: US 2018/0243938 A1
(43) Pub. Date: Aug. 30, 2018

(54) PROCESS FOR THE MANUFACTURE OF WOOD COMPOSITE MATERIALS AS WELL AS WOOD COMPOSITE MATERIALS OBTAINABLE BY THE PROCESS

(71) Applicant: AVALON Industries AG, Zug (CH)

(72) Inventors: Francois BADOUX, Unteraegeri (CH); Stefan KRAWIELITZKI, Holzhaeusern (CH); Mariangela MORTATO, Basel Stadt (CH); Reto FREI, Evilard (CH); Marie-Christine LAGEL, Mommenheim (FR); Christopher HOLMES, Nunningen (CH)

(73) Assignee: AVALON Industries AG, Zug (CH)

(21) Appl. No.: 15/904,811

(22) Filed: Feb. 26, 2018

(30) Foreign Application Priority Data
Feb. 27, 2017 (EP) 17158248.9

(51) Int. Cl. B27N 3/00; C08G 14/06; C08J 5/04; C08K 5/21; C08K 5/3492; C08K 5/405; C07D 307/48

(52) U.S. Cl. CPC B27N 3/002; C08G 14/06; C08J 5/045; C07D 307/48; C08K 5/34922; C08K 5/405; C08K 5/21

(57) ABSTRACT

A process for the manufacture of wood composite materials includes the steps of: preparation of a thermally curable resin by reacting a polycondensation-capable phenolic compound and/or an amino plastic forming agent with 5-hydroxymethylfurfural (HMF) under conditions leading to the formation of polycondensation products, bringing the resin into contact with lignocellulose-containing material, and curing the resin with formation of the wood composite material. The 5-hydroxymethylfurfural includes at least one HMF oligomer. Further, wood composite materials are obtainable by the process.

### CROSS REFERENCE TO RELATED APPLICATIONS

Applicant claims priority under 35 U.S.C. § 119 of European Application No. 17158248.9 filed Feb. 27, 2017, the disclosure of which is incorporated by reference.

### BACKGROUND OF THE INVENTION

### 1. Field of the Invention

The present invention relates to a process for the manufacture of wood composite materials. In particular, the invention relates to a process for the manufacture of wood composite materials including the steps of:
preparation of a thermally curable resin by reacting a polycondensation-capable phenolic compound and/or an amino plastic forming agent with 5-hydroxymethylfurfural (HMF) under conditions leading to the formation of polycondensation products,
bringing the resin into contact with lignocellulose-containing material, and
curing the resin with formation of the wood composite material.

The invention likewise relates to the wood composite materials obtainable by the process.

### 2. Description of the Related Art

Wood composite materials are typically manufactured from lignocellulose-containing material, such as wood shavings, wood fibers or wood chips and a thermally curable resin. Amino resins with the aminoplastic forming agents urea, melamine and dicyandiamide, phenol resins or aminophenol resins may be mentioned as examples of thermally curable resins. The wood composite materials are usually obtained by bringing the lignocellulose material into contact with the resins and pressing at elevated temperatures, wherein the resins are cured, which is associated with a cross-linking.

The wood composite materials produced with the resins find their practical application by virtue of their good mechanical properties, such as good internal bond strength (IB), insensitivity to moisture, especially steam, in particular in furniture, where it is used in the form, for example, of panels of plywood, wood-fiber composite, chipboard and multilayer board. These items are used predominantly in interior rooms. For this reason, it is important that the wood composite materials do not emit any harmful compounds. These harmful compounds originate mainly from the resins used.

Thermally curable resins are usually obtained by the polycondensation of phenolic compounds and/or aminoplastic forming agents with reactive carbonyl compounds, especially aldehydes. On the basis of its high reactivity, mainly formaldehyde, which must be classified as hazardous to health, is used for the polycondensation. To promote the reaction, it is frequently carried out with an excess of formaldehyde, which is highly volatile, and so the resins have a high content of free formaldehyde. The formaldehyde emission of the resins and of the wood composite materials manufactured with it is therefore likewise high.

Because of the hazard potential, efforts have been made for years to reduce the content of formaldehyde. One measure in this respect is to replace formaldehyde in the manufacture of the resins by other reactive compounds. 5-hydroxymethylfurfural (HMF) has already been identified as a highly promising candidate for this purpose, because it has the ability to form cross-linking bonds, is sparingly volatile as well as practically nontoxic, and can be obtained from renewable raw materials.

U.S. Pat. No. 4,524,164 A relates to a formaldehyde-free thermally curable adhesive resin and to a process for use of the adhesive resin for the bonding of lignocellulose-containing material, in order to form products such as plywood and chipboard panels. Firstly a liquid meltable resin is prepared, by heating an aqueous sugar or starch solution in the presence of a cross-linking agent, which is selected from urea or a phenol or mixtures of these, together with an inorganic acid or its ammonium salt and a metal-ion catalyst. The meltable resin is then mixed with an organic acid anhydride and applied on the surface of the lignocellulose-containing material. For forming of products such as plywood or chipboard panels, the mixture is then exposed to heat and pressure.

In the trade magazine European Journal of Wood Products, an HMF-modified urea-formaldehyde resin is described. For the manufacture of this resin up to approximately 30 wt % of the formaldehyde was replaced by purified, crystalline HMF (N. Esmaeili et al., DOI 10.1007/s0017-016-1072-8). Chipboard panels manufactured with this resin have an internal bond strength (IB) of ≥0.35 N/mm², as is currently required to fulfill the minimum standards for panels in interior rooms in accordance with European Standard NEN EN 319. Nevertheless, it is disadvantageous that the resin and chipboard panels manufactured from it still contain considerable quantities of toxic formaldehyde.

### SUMMARY OF THE INVENTION

Accordingly, the object of the present invention consists in the elimination of the above-mentioned disadvantages.

These and other objects are accomplished by a process for the manufacture of wood composite materials according to a first aspect of the invention. According to a further aspect, the present invention relates to a wood composite material that is obtainable via the process.

The process for the manufacture of wood composite materials includes the steps of:
preparation of a thermally curable resin, by reacting a polycondensation-capable phenolic compound and/or an amino plastic forming agent with 5-hydroxymethylfurfural (HMF) under conditions leading to the formation of polycondensation products,
bringing the resin into contact with lignocellulose-containing material, and
curing the resin with formation of the wood composite material.

The process is characterized in that the 5-hydroxymethylfurfural comprises at least one HMF oligomer.

It has been found possible to dispense with formaldehyde completely for the manufacture of wood composite materials, provided HMF that contains HMF oligomers is used in the preparation step for the polycondensation. It is assumed that the HMF oligomers are more reactive than HMF monomers, which permits a process for the manufacture of wood composite materials without the use of formaldehyde.

The occurrence of water-soluble linear and branched HMF oligomers in solutions of HMF is known, for example, from DE 10 2014 112 240 A1. The formation of the HMF oligomers may be followed using HPLC analyses, for example.

In contrast to HMF monomers, compounds of at least two linked HMF units/monomers are designated as HMF oligomers within the meaning of the present invention. Usually, HMF oligomers are understood as compounds with a molar mass of up to 3000 g/mol. HMF oligomers with a low molar mass are suitable in particular for the process. Such HMF oligomers under the selected reaction conditions are present in soluble or at least in dispersed form in the selected solvent. In this connection, the transition between dissolved and dispersed form may be continuous. Accordingly, a distinction in this respect will not be made in the present invention.

The polycondensation for the preparation of the thermally curable resins is undertaken in a way known in itself. Solvents suitable for the reaction as well as suitable reaction conditions such as reaction temperature and pH are in principle known to the person skilled in the art. Preferably, the reaction is carried out in an aqueous solvent.

In this connection, it is self-evident for the person skilled in the art that the at least one HMF oligomer may be present in a mixture of HMF oligomers of various lengths and/or various degrees of cross-linking. In addition, it is possible, by the selection of an HMF oligomer or by the selection of a combination of different HMF oligomers, to match the properties of the resin resulting from the preparation step selectively to the technical purpose of use.

### BRIEF DESCRIPTION OF THE DRAWINGS

Other objects and features of the invention will become apparent from the following detailed description considered in connection with the accompanying drawings. It is to be understood, however, that the drawings are designed as an illustration only and not as a definition of the limits of the invention.

In the drawings,

FIG. 1 shows a proposed mechanism of the carbon-carbon bond formation under acidic conditions on the basis of dimerization of two HMF molecules, and

FIG. 2 shows a proposed mechanism of the carbon-carbon bond formation under basic conditions on the basis of dimerization of two HMF molecules.

### DETAILED DESCRIPTION OF PREFERRED EMBODIMENTS

According to one advantageous configuration of the process, the reaction for the preparation of the resins is carried out at temperatures in the range of 40° C. to 140° C., preferably in the range of 50° C. to 140° C., more preferably in the range of 60° C. to 100° C., particularly preferably in the range of 80° C. to 100° C. In principle, the temperature for carrying out the polycondensation may be varied within a broad range. It has been observed, however, that more reactive resins can be obtained by the reaction at higher temperatures. Particularly highly reactive resins, which need short press times for curing during the subsequent formation of the wood materials, may be obtained at temperatures in the range of 80° C. to 100° C. This result was unexpected because heretofore it was assumed that increasing decomposition of HMF would already have taken place starting from temperatures above 50° C.

According to a further advantageous configuration of the process, the mole ratio of the HMF quantity used to the total quantity of phenolic compound and/or aminoplastic forming agent is 0.20:1 to 3:1; preferably the mole ratio is 0.3:1 to 1:1; particularly preferably the mole ratio is 0.45:1 to 0.70:1. In principle, the mole ratio of the HMF quantity used to the total quantity of phenolic compound and/or aminoplastic forming agent may be varied over a broad range. A molar excess of HMF may also be advantageous. A suitable molar ratio for the respective phenolic compound, the respective aminoplastic forming agent or for a mixture of phenolic compound and aminoplastic forming agent can be easily determined by the person skilled in the art.

According to a further advantageous configuration of the process, the proportion of HMF oligomer is 0.05 wt % to 10 wt % relative to the total quantity of HMF used; preferably the proportion of HMF oligomer is 0.1 wt % to 8 wt % relative to the total quantity of HMF used; particularly preferably the proportion of HMF oligomer is 2 wt % to 4 wt % relative to the total quantity of HMF used. Due to the high reactivity, even small quantities of HMF oligomer are sufficient to prepare reactive resins. It is self-evident for the person skilled in the art that higher proportions of HMF oligomers may also be used. The invention likewise comprises that the HMF oligomer makes up to or up to almost 100 wt % relative to the total quantity of HMF used.

According to a further advantageous configuration of the process, the HMF oligomer has 2 to 20 units, preferably 2 to 10 units, particularly preferably 2 to 4 units. HMF oligomers with 2 to 10 units are readily water-soluble under moderate conditions, meaning room temperature and normal pressure, and so the HMF oligomers can be used without problems for a polycondensation in an aqueous medium. HMF oligomers with 2 to 4 units have an improved water solubility. HMF oligomers with 2 units are particularly readily water-soluble and therefore particularly well suited for the reaction.

According to a further preferred configuration of the process, the HMF oligomer used for the reaction is a carbon-linked HMF oligomer.

Within the meaning of the present invention, HMF oligomers are designated as carbon-linked HMF oligomers, provided at least two HMF units are linked by a carbon-carbon bond with involvement of an aromatically bound carbon atom at position 3 or 4 of the furan ring of one of the two HMF units. In particular, the carbon-linked HMF oligomer contains at least one first unit, the aldehyde-group carbon atom of which is linked with an aromatically bound carbon atom of the furan ring of a second unit.

The inventors have found that, besides HMF oligomers that result from the linking of aldehyde and/or hydroxyl groups of the HMF units and that have the corresponding ether, hemiacetal and/or acetal bonds, HMF oligomers in which units are linked by a carbon-carbon bond are formed both under acidic conditions and under basic conditions. As an example, these bonds may be formed during an electrophilic attack of an aldehyde group of a first HMF monomer or of an HMF unit of an HMF oligomer at the carbon atom in position 3 or 4 of a furan ring of a second HMF monomer or of an HMF unit of an HMF oligomer.

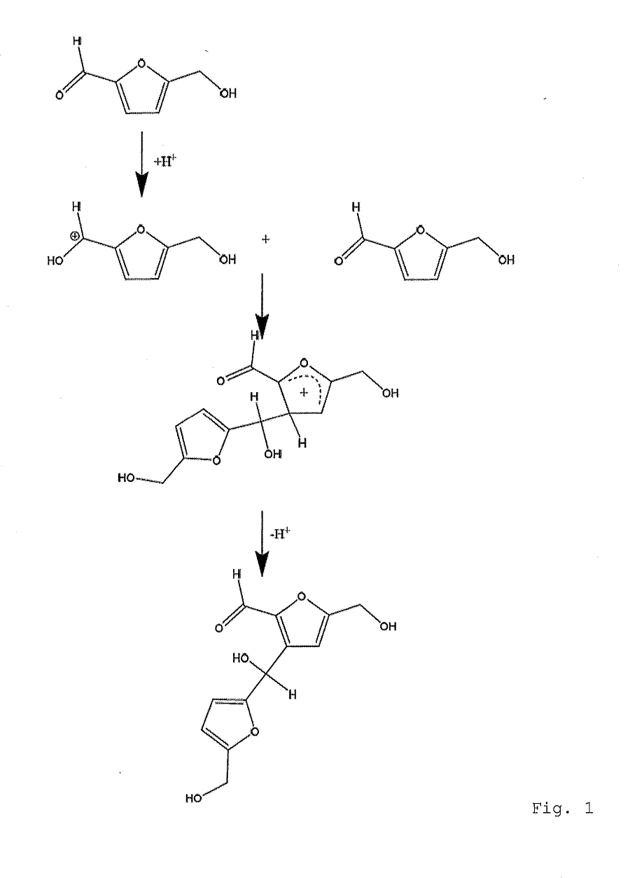

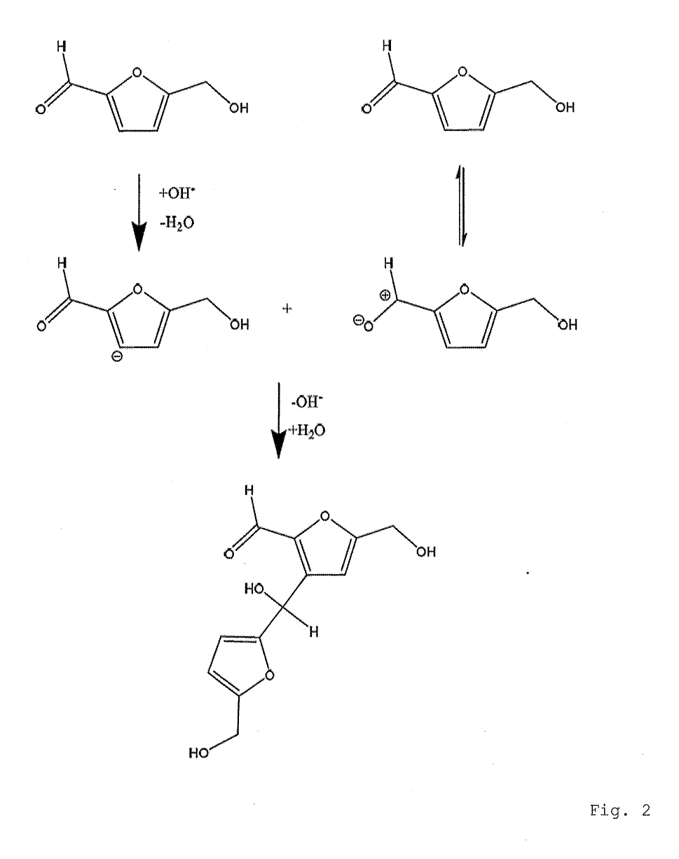

The mechanisms proposed for the HMF oligomer formation under acidic conditions and under basic conditions are presented in FIGS. 1 and 2. From these mechanisms, it is evident among other facts that HMF oligomers having a link via a carbon-carbon bond at the same time have more free functional aldehyde and/or hydroxyl groups than do HMF oligomers in which the bond is formed merely via aldehyde and/or hydroxyl groups of the HMF. Hereby very reactive HMF oligomers are obtained, which have additional cross-linking capabilities.

The carbon-linked HMF oligomers may contain, besides the bond linked with involvement of an aromatically bound carbon atom, also other bonds, such as ether, hemiacetal and/or acetal bonds. To increase the reactivity of the resulting resin, it is sufficient when two of the HMF units are already linked with involvement of an aromatically bound carbon atom. In particular, carbon-linked HMF oligomers with 2 units contain a relatively high proportion of free functional, reactive groups per HMF oligomer. The carbon-linked HMF oligomer may also have several such carbon-carbon links.

Furthermore, besides the carbon-linked HMF oligomers, still further HMF oligomers with ether, hemiacetal and/or acetal bonds may be present. Due to the high proportion of free functional groups, even small quantities of carbon-linked HMF oligomer are sufficient to prepare very reactive oligomers. The invention likewise comprises that the carbon-linked HMF oligomer makes up to or up to almost 100 wt % relative to the total quantity of HMF oligomer.

The polycondensation-capable phenolic compound or the aminoplastic forming agent or both may be such that are usually used for the manufacture of thermally curable resins.

In this connection, all hydroxyl-group-containing aromatic compounds that have, in the aromatic part, at least one carbon atom that is amenable to a nucleophilic addition reaction between phenolic compounds and the HMF, may be regarded in principle as polycondensation-capable compounds.

Advantageously, the polycondensation-capable phenolic compound is phenol, lignin, a phenolic compound derived from lignin, resorcinol, hydroquinone, hydroxyquinone, pyrocatechol, phloroglucinol or a mixture of at least two of these compounds.

Advantageously, the aminoplastic forming agent is urea, melamine, substituted melamine, substituted urea, acetylene diurea, guanidine, thiourea, thiourea derivative, diaminoalkane, diamidoalkane or a mixture of at least two of these aminoplastic forming agents.

In this connection, still further phenolic compounds or aminoplastic forming agents or both may be present besides the cited components.

Depending on the phenolic compound and/or the aminoplastic forming agent, the pH may be varied over a broad range in the preparation step. For example, the pH may lie in the range of 6 to 10, preferably in the range of 7 to 8.5.

According to a further advantageous configuration of the process, the preparation step is carried out in a solution until the solution has attained a desired viscosity or the reaction has ended. Preferably, the preparation step is carried out until the solution has reached a viscosity of over 200 mPa·s, particularly preferably until the solution has reached a viscosity of over 450 mPa·s.

Advantageously, very reactive thermally curable resins are prepared which, by curing in contact with a lignocellulose-containing material, yield wood composite materials with very good mechanical properties.

Preferably, the thermally curable resin comprises at least one polymer obtained by polycondensation of phenolic compounds and/or aminoplastic forming agents with 5-hydroxymethylfurfural (HMF), wherein the polymer is a polycondensation product of a phenolic compound and/or an aminoplastic forming agent with an HMF oligomer.

Within the meaning of the present invention, products of the polycondensation are understood under the term polymer. The polymers are usually water-insoluble.

Particularly preferably, the polymer is a polycondensation product of a phenolic compound and/or an aminoplastic forming agent with a carbon-linked HMF oligomer, which contains at least one first HMF unit linked to an aromatically bound carbon of a second HMF unit.

The solids content of the resin obtained in the preparation step may be varied over a broad range. The solids content is at least 40 wt %. Preferably, the solids content of the resin lies in the range of 45 wt % to 80 wt %, particularly preferably between 50 wt % and 70 wt %.

According to a further advantageous configuration of the process, the process includes at least one further step, which makes available, for the step of preparation of the thermally curable resin, 5-hydroxymethylfurfural comprising at least one HMF oligomer.

Preferably the make-available step includes exposing a more or less pure solution of HMF monomers and/or HMF oligomers to conditions that lead to the formation of HMF oligomers. The inventors have found that aqueous HMF solutions that were prepared, for example, from crystalline HMF with water, age accompanied by formation of the HMF oligomers. In this connection, the quantity and the molecular mass of the HMF oligomers may be determined using analytical means familiar to the person skilled in the art, such as high-performance liquid chromatography (HPLC) and nuclear magnetic resonance (NMR) spectroscopy.

The formation of HMF oligomers under moderate conditions, meaning at normal pressure and room temperature, may last in the range of hours, days or weeks.

Particularly preferably, the conditions to which the HMF solution is exposed comprise alkalinization or acidification of the solution. Likewise, the conditions particularly preferably comprise the heating of the solution, if necessary in combination with acidification or alkalinization. The aging process can be accelerated by acidification, alkalinization and heating.

A particularly preferred variant of the make-available step includes making 5-hydroxymethylfurfural available that comprises at least one HMF oligomer by treatment of an aqueous suspension of cellulose-containing biomass and/or an aqueous carbohydrate solution of at least one hexose and/or one aqueous 5-hydroxymethylfurfural solution under hydrothermal conditions.

The treatment of biomass, such as plant-based raw materials, of carbohydrates or of compounds derived from carbohydrates under hydrothermal conditions for the production of 5-HMF is known. The treatment provides for exposing the starting material to pressure and elevated temperature in aqueous medium. During the treatment of an aqueous suspension of cellulose-containing biomass and/or of an aqueous carbohydrate solution of at least one hexose and/or one aqueous 5-hydroxymethylfurfural solution under hydrothermal conditions, HMF oligomers are formed.

Cellulose-containing biomass, which frequently accumulates as a waste product of the agricultural producers, is particularly preferred because of its low cost factor. Preferred hexoses are fructose or glucose; in particular, they may be fructose or mixtures of fructose and glucose.

Preferred hydrothermal conditions are saturated-steam pressure and temperatures of 150° C. to 250° C. These conditions have the advantage that the formation of HMF oligomers is completed within minutes to a few hours, depending on the starting material.

Preferably, the make-available step is carried out until the desired quantity of HMF oligomer is reached or until the reaction has stopped.

Preferably, the HMF, which comprises at least one HMF oligomer, is present in aqueous solution at the end of the make-available step. It is further preferable to influence the content, the size and/or the concentration of the oligomer or of the oligomers. Particularly preferably, the content of the oligomer or of the oligomers is influenced by subjecting the solution obtained in the make-available step to a filtration on at least one filtration means. The treatment of an aqueous HMF solution after a hydrothermal carbonization is described in DE 10 2014 112 240 A1, for example:

According to a further advantageous configuration of the process, the lignocellulose-containing material, which is brought into contact with the resin, comprises wood shavings, wood fibers, wood flocks, wood chips, wood particles, wood strips, wood flakes and boards as well as mixtures of these items. The lignocellulose-containing material may be varied within a wide range with respect to the large number of different wood composite materials.

The bringing of the resin into contact with lignocellulose-containing material may take place by methods known to the person skilled in the art. Usually the bringing into contact is performed in dependence on the nature of the resin and the configuration of the lignocellulose-containing material. For example, the bringing into contact may be performed by spraying, brushing on, mixing by mechanical stirring or roller application.

According to a further advantageous configuration of the process, the lignocellulose-containing material is brought into contact in a quantity of 2 wt % to 20 wt %, preferably in a quantity of 5 wt % to 15 wt % of resin relative to the weight of the dry lignocellulose-containing material. The resin quantity may be varied within a wide range depending on the configuration of the lignocellulose-containing material and the requirements applicable to the wood composite material. In addition, it may be advantageous to bring mixtures of two or more resins into contact with the lignocellulose-containing material.

According to a further advantageous configuration of the process, the curing of the resin comprises a pressing of the lignocellulose-containing material with the resin. Usually pressures of 1 mPa to 30 mPa are used.

Preferably, the pressing takes place at a temperature in the range of 120° C. to 250° C., particularly preferably in the range of 210° C. to 230° C. Due to the reactivity of the resins, even as little as a few minutes is sufficient for production of wood materials with good mechanical properties. Preferably, the press time lies in the range of 3 minutes to 10 minutes; particularly preferably, the press time lies in the range of 5 minutes to 8 minutes. A short press time is advantageous from both the production-engineering and economic viewpoints.

If desired, the cross-linking ability of the resins may be enhanced by adding a curing agent to the resins. Preferably the quantity of curing agent is in the range of 2 wt % to 7 wt % relative to the quantity of resin, particularly preferably in the range of 2 wt % to 5.5 wt % relative to the quantity of resin, quite particularly preferably in the range of 2 wt % to 3 wt % relative to the quantity of resin. Curing agents may be in particular hexamethylenetetramine or ammonium salts such as ammonium sulfate. The reactivity of the HMF oligomers is so high, however, that merely very small quantities of curing agent have to be used in order to obtain resins with a high cross-linking ability. It may also be possible to dispense with a curing agent completely.

The obtained wood composite materials may finally be post-treated for stabilization in a drying oven or wood dryer at temperatures in the range of 10° C. to 100° C. under controlled atmosphere. Such may comprise, for example, a relative humidity in the range of 40% to 70%.

According to a further aspect, the present invention relates to a wood composite material that is obtainable via the process described above.

One advantage of the manufacture of a wood composite material with the process according to the invention is that the wood composite materials are formaldehyde-free and can be manufactured on the basis of natural, renewable raw materials, and in the process have a very good resistance to moisture, especially steam. A further advantage of the process is that, due to the reactivity of the HMF oligomers, short press times in the range of minutes are sufficient to obtain a wood composite material with very good mechanical properties. This feature is highly desirable from economic viewpoints and with respect to the industrial production of wood composite materials.

According to a further advantageous configuration of the wood composite material, the wood composite material is a panel of plywood, wood fiber composite, chipboard or multilayer board with an internal bond strength (IB) of 0.35 N/mm².

An advantage of the wood composite material is that it exceeds the requirements of the minimum standard in accordance with European Standard NEN EN 319, as may also be inferred from the following examples.

The following examples serve merely as the explanation of the invention and are not intended to restrict it in any way.

### Example 1

### Manufacture of Chipboard Panels

### a) Preparation of an HMF Solution Containing HMF Oligomers:

A 16% aqueous solution of crystalline HMF was simultaneously concentrated and aged by reducing the volume in a rotary evaporator at 45° C. and 30 mbar until the concentration of HMF was 50 wt % relative to the solution.

### b) Preparation of Urea-HMF Resins and Comparison of the Properties:

Two resins differing in their mole ratio of urea to HMF were prepared. A first resin, denoted in the following by UH(1:0.5), was prepared with a ratio of urea to HMF of 1:0.5. A second resin, denoted in the following by UH(1:0.25), was prepared with a ratio of urea to HMF of 1:0.25. The solids content of the resins was approximately 58%. For both resins, 400 mL of the 50% HMF solution from a) was used. For both resins, the urea was reacted with HMF at a pH of 2, for 2.5 hours and a temperature of 90° C. at first and then for several hours at a temperature of 20° C. In the process, the change of the viscosity of the resins was observed.

TABLE 1
Increase of viscosity as a function of time

Viscosity [mPa · s]

| Time [hours] | UH(1:0.5) | UH(1:0.25) |
|---|---|---|
| 4 | 470 | — |
| 24 | 1275 | 58 |
| 48 | — | 60 |
| 120 | — | 65 |
| 144 | — | 65 |
| 168 | — | 65 |

### c) Pressing of Wood Shavings to Chipboard Panels:

Resin UH(1:0.5) with a viscosity of 1275 mPa·s and resin UH(1:0.25) with a viscosity of 65 mPa·s were used for the subsequent pressing of wood shavings. The resins were mixed respectively with the wood shavings and with hexamethylenetetramine and then pressed at 220° C. for the production of panels measuring 250 mm×250 mm×16 mm. The loading of the dry wood was 10 wt % resin solid relative to the quantity of wood. In order to test the influence of various press times and various quantities of curing agent, several panels were produced with variation of the times and of the quantities of hexamethylenetetramine. The values obtained for the chipboard panels with the two resins UH(1:0.5) and UH(1:0.25) are presented in Table 2.

For comparison, a third resin, UH45(1:0.5), was produced by reacting the components of the resin UH(1:0.5) at a lower temperature of 45° C. The resin UH45(1:0.5) was also used for pressing of wood shavings to chipboards measuring 250 mm×250 mm×16 mm. The values obtained for these chipboard panels are also presented in Table 2.

The comparison of the panels produced with the resins showed that, in principle, better values of internal bond strength are obtained at a longer press time.

With a mole ratio of urea to HMF of 1:0.5, the panels 3 and 4 attained the high values of 52 N/mm² and 55 N/mm². These values can be attributed to a press time of 7.5 minutes in association with a high temperature of 90° C. for preparation of the resins.

The panels 1 and 2 as well as 5 and 6 illustrate the influence of temperature during the preparation of the resins.

Even panels produced with smaller quantities of HMF yield a satisfactory result when the press time is prolonged, as shown by panels 7 to 10.

As regards the curing agent, it was found that different quantities of curing agent are slightly noticeable to unnoticeable, provided the panels were produced with a certain proportion of HMF, as shown by panels 3 to 6. The panels 7 and 10, with lower proportions of HMF, are clearly influenced more strongly by the quantity of curing agent. The values illustrate that, as a consequence of the positive properties of the HMF oligomers used, the needed quantities of curing agent can be reduced drastically to obtain nonetheless products with identical or comparable internal bond strength.

Internal Bond Strength (IB) in Accordance with NF EN 319 (AFNOR 1993):

The internal bond strength in [N/mm²] is expressed by the following formula:

$$IB = \frac{Fmax}{a \times b} ,$$

where Fmax is the force at break, a the width and b the length of the panel.

For chipboard and fiberboard panels with a thickness in the range of 13 mm to 20 mm, NF EN 319 (AFNOR 1993) specifies an internal bond strength of ≥0.35 N/mm².

The panels for investigation of the internal bond strength were obtained by cutting out of the panels produced under c). Their size was 50 mm×50 mm. Prior to the cutting, the panels were stabilized in a dryer at 20° C. and a relative humidity of 65%.

The panels were fastened to a backing by means of a hot-melt adhesive. The determination of the internal bond strength was performed mechanically, perpendicular to the plane of the panels, in accordance with NF EN 319 (AFNOR 1993).

TABLE 2
Parameters of the production of chipboard panels, and properties of the chipboard panels

| | | | | | | | | | Internal |
|---|---|---|---|---|---|---|---|---|---|
| | | | | Mole | | | | | bond |
| | | Synthesis | | ratio of | Press | Press | Curing | | strength |
| | | temperature | Viscosity | urea to | temperature | time | agent | Density | (IB) |
| Panel | Resin | [° C.] | [mPa · s] | HMF | [° C.] | [min] | [%] | [kg/m²] | [N/mm²] |
| 1 | UH45(1:0.5) | 45 | 382 | 1:0.5 | 220 | 5.5 | 5 | 733 | 0.27 |
| 2 | UH45(1:0.5) | 45 | 382 | 1:0.5 | 220 | 5.5 | 2.5 | 729 | 0.21 |
| 3 | UH(1:0.5) | 90 | 1275 | 1:0.5 | 220 | 7.5 | 5 | 717 | 0.55 |
| 4 | UH(1:0.5) | 90 | 1275 | 1:0.5 | 220 | 7.5 | 2.5 | 718 | 0.52 |
| 5 | UH(1:0.5) | 90 | 1275 | 1:0.5 | 220 | 5.5 | 5 | 715 | 0.43 |
| 6 | UH(1:0.5) | 90 | 1275 | 1:0.5 | 220 | 5.5 | 2.5 | 718 | 0.43 |
| 7 | UH(1:0.25) | 90 | 65 | 1:0.25 | 220 | 7.5 | 5 | 714 | 0.44 |
| 8 | UH(1:0.25) | 90 | 65 | 1:0.25 | 220 | 6.5 | 5 | 715 | 0.39 |
| 9 | UH(1:0.25) | 90 | 65 | 1:0.25 | 220 | 5.5 | 5 | 712 | 0.31 |
| 10 | UH(1:0.25) | 90 | 65 | 1:0.25 | 220 | 7.5 | 2.5 | 713 | 0.36 |

All features of the invention can be essential to the invention both individually as well as in any combination whatsoever with one another.

Although several embodiments of the present invention have been shown and described, it is to be understood that many changes and modifications may be made thereunto without departing from the spirit and score the invention.

What is claimed is:

1. A process for manufacturing a wood composite material comprising:
(a) reacting in a reaction step at least one of a polycondensation-capable phenolic compound and an aminoplastic forming agent with 5-hydroxymethylfurfural under conditions leading to the formation of polycondensation products to prepare a thermally curable resin;
(b) bringing the resin into contact with a lignocellulose-containing material; and
(c) curing the resin to form the wood composite material;
wherein the 5-hydroxymethylfurfural comprises at least one hydroxymethylfurfural oligomer.

2. The process according to claim 1, wherein the reaction step is carried out at temperatures in the range of 40° C. to 140° C.

3. The process according to claim 1, wherein the proportion of the at least one hydroxymethylfurfural oligomer is 0.05 wt % to 10 wt % relative to a total quantity of the hydroxymethylfurfural used.

4. The process according to claim 1, wherein the at least one hydroxymethylfurfural oligomer has 2 to 20 units.

5. The process according to claim 1, wherein the at least one hydroxymethylfurfural oligomer is a carbon-linked hydroxymethylfurfural oligomer.

6. The process according to claim 1, wherein the 5-hydroxymethylfurfural is reacted with a polycondensation-capable phenolic compound in the reaction step and the polycondensation-capable phenolic compound is phenol, lignin, a phenolic compound derived from lignin, resorcinol, hydroquinone, hydroxyquinone, pyrocatechol, phloroglucinol or a mixture of at least two of these compounds.

7. The process according to claim 1, wherein the 5-hydroxymethylfurfural is reacted with an aminoplastic forming agent in the reaction step and the aminoplastic forming agent is a member selected from the group consisting of urea, melamine, substituted melamine, substituted urea, acetylene diurea, guanidine, thiourea, thiourea derivative, diaminoalkane, diamidoalkane and a mixture of at least two of these members.

8. The process according to claim 1, wherein the reaction step is carried out in a solution until the solution has attained a selected viscosity or until the reaction has ended.

9. The process according to claim 1, further comprising performing at least one further process step so that the reaction step yields 5-hydroxymethylfurfural comprising at least one hydroxymethylfurfural oligomer.

10. The process according to claim 9, wherein the 5-hydroxymethylfurfural is made available by treatment of at least one of an aqueous suspension of cellulose-containing biomass, an aqueous carbohydrate solution of at least one hexose, and an aqueous 5-hydroxymethylfurfural solution under hydrothermal conditions.

11. The process according to claim 1, wherein the lignocellulose-containing material is a member selected from the group consisting of wood shavings, wood fibers, wood flocks, wood chips, wood particles, wood strips, wood flakes, boards, and mixtures of said members.

12. The process according to claim 1, wherein the lignocellulose-containing material is brought into contact in a quantity of 2 wt % to 20 wt % of the resin relative to the weight of the dry lignocellulose-containing material in dry form.

13. The process according to claim 1, wherein the lignocellulose-containing material is brought into contact in a quantity of 5 wt % to 15 wt % of the resin relative to the weight of the lignocellulose-containing material in dry form.

14. The process according to claim 1, wherein the curing of the resin comprises a pressing of the lignocellulose-containing material with the resin.

15. A wood composite material obtained by a process comprising:
(a) reacting in a reaction step at least one of a polycondensation-capable phenolic compound and an aminoplastic forming agent with 5-hydroxymethylfurfural under conditions leading to the formation of polycondensation products to prepare a thermally curable resin;
(b) bringing the resin into contact with a lignocellulose-containing material; and
(c) curing the resin to form the wood composite material;
wherein the 5-hydroxymethylfurfural comprises at least one hydroxymethylfurfural oligomer.

16. The wood composite material according to claim 15, wherein the wood composite material is a panel of plywood, wood-fiber composite, chipboard or multilayer board and has an internal bond strength (IB) of ≥0.35 N/mm², determined in accordance with NF EN 319 (AFNOR 1993).
